(19) United States
(12) Patent Application Publication
(10) Pub. No.: US 2024/0150275 A1
(43) Pub. Date: May 9, 2024

(54) SYNERGISTIC ANTIFOULANT COMPOSITIONS AND METHODS OF USING THE SAME

(71) Applicant: ECOLAB USA Inc., St. Paul, MN (US)

(72) Inventor: Meng KONG, Shanghai (CN)

(73) Assignee: ECOLAB USA Inc., St. Paul, MN (US)

(21) Appl. No.: 18/491,438

(22) Filed: Oct. 20, 2023

(30) Foreign Application Priority Data
Oct. 25, 2022 (CN) 202211309396.9

(51) Int. Cl. C07C 67/62

(52) U.S. Cl. CPC C07C 67/62

(57) ABSTRACT

Antifouling compositions are provided. The antifouling compositions include a dispersing agent including an imidazoline, a solvator including a phthalate, and a promoter agent including an acylamide. Methods of mitigating fouling caused by the polymerization of monomers using the compositions of the disclosure are also provided. The methods of mitigating fouling of monomers include a step of adding a composition of the disclosure to the monomer. In some instances, the monomer is an ethylenically unsaturated monomer, such as (meth)acrylic acid, methyl methacrylate, an acrylic acid ester, methacrylamide sulfate, vinyl acetate, acrylonitrile, acrolein, an acrylate, a methacrylate, 1,3-butadiene, styrene, isoprene, and any combination thereof. Methods of preparing the antifouling compositions are also provided.

### FIELD OF THE INVENTION

The present disclosure generally relates to antifoulant compositions that include a blend of a dispersing agent, a solvator agent and a promoter agent and methods of using the same. More particularly, the present disclosure relates to compositions that include a dispersing agent comprising an imidazoline, a solvator comprising a phthalate and a promoter agent comprising an acylamide, useful for mitigating fouling of ethylenic unsaturated monomers. The present disclosure further relates to methods of preparing compositions having a dispersing agent comprising an imidazoline, a solvator comprising a phthalate and a promoter agent comprising an acylamide.

### BACKGROUND

The manufacture of ethylenically unsaturated monomers typically comprises three stages: reaction, recovery, and purification. Distillation operations at elevated temperatures are often involved in the recovery and the purification stages. Ethylenically unsaturated monomers, such as vinyl acetate, acrylate, and methacrylate monomers, can be present in processing streams or in refined products made by various chemical industrial processes. However, these monomer types may undesirably polymerize through radical polymerization especially at elevated temperature and when polymerization initiators are present. As a result, solid deposits of polymer can form on the surface of the process equipment during industrial manufacture, processing, handling, or storage. The resulting polymers can be problematic and lead to equipment “fouling” and product contamination. Accordingly, this can necessitate treating the apparatus to remove the polymer, or may necessitate processing steps to remove the polymer from compositions streams or stored compositions. These undesirable polymerization reactions result in a loss in production efficiency because they consume valuable reagents and additional steps may be required to clean equipment and/or to remove the undesired polymers.

The premature polymerization of these monomers is generally controlled by dosing polymerization inhibitors capable of reducing the premature polymerization of the monomers. Conventional polymerization inhibitors include stable free radicals that can effectively scavenge carbon-centered radicals. Conventional polymerization inhibitors, such as 4-hydroxy-2,2,6,6-tetramethylpiperidin-1-oxyl (HTEMPO) and 4-oxo-2,2,6,6-tetramethylpiperidin-1-oxyl (OTEMPO), generally degrade and lose their efficacy as polymerization inhibitors under acidic environments. Thus, there is a need to develop new polymerization inhibitors, particularly inhibitors that are stable under acidic conditions.

Methyl methacrylate plants are troubled by fouling issues which can occur in methacrylic acid (MAA) production, esterification units, purification units and spent acid units. Phenothiazine (PTZ) solution is typically prepared at a low concentration because of poor solubility, however, a concentrated formula is needed to improve antifouling applications.

### BRIEF SUMMARY

Compositions for mitigating fouling caused by polymerization of monomers are disclosed herein. The compositions include a dispersing agent that is an imidazoline, a solvator that is a phthalate and a promoter agent that is an acylamide. In some embodiments, the compositions are useful for mitigating fouling of ethylenically unsaturated monomers including methacrylic acid, methyl methacrylate, acrylic acid, acrylic acid esters, methacrylamide sulfate, vinyl acetate, acrylonitrile, acrolein, acrylates, methacrylates, 1,3-butadiene, styrene, isoprene, and combinations thereof.

In some aspects, the dispersing agent comprises an imidazoline of formula (I):

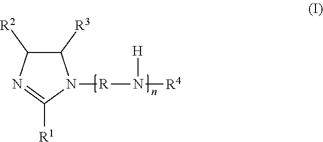

or a derivative thereof, wherein R is C₁-C₃₀ alkylene, wherein R¹, R² and R³ are each independently selected from the group consisting of H, C₁-C₃₀ alkyl, C₂-C₃₀ alkenyl, C₆-C₃₀ aryl, C₁-C₃₀ alkyl-C₆-C₃₀ aryl, C₆-C₃₀ aryl-C₁-C₃₀ alkyl, C₁-C₃₀ aminoalkyl, and C₆-C₃₀ aminoaryl, wherein R⁴ is selected from the group consisting of hydrogen, —(CH)COOH, —CHCH(CH)COOH, imidazoline, C₁-C₃₀ alcohol, C₁-C₃₀ ester, C₁-C₃₀ amide, and C₁-C₃₀ alkyl-C₆-C₃₀ aryl, and wherein n is 0, 1, 2, 3, 4, 5, 6, 7, 8, or 9. In some embodiments, R is a C₁-C₁₀ alkylene. In some embodiments, R⁴ is selected from the group consisting of hydrogen, —CH₂COOH, —(CH₂)₂COOH, —CH₂CH(CH₃)COOH, imidazoline, C₁-C₃₀ alcohol, and C₁-C₃₀ alkyl-C₆-C₂₀ aryl.

In some embodiments, R is C₁-C₁₀ alkylene, wherein R¹, R² and R³ are each independently selected from the group consisting of H, C₁-C₁₀ alkyl, C₂-C₁₀ alkenyl, C₆-C₂₀ aryl, C₁-C₁₀ alkyl-C₆-C₂₀ aryl, C₆-C₂₀ aryl-C₁-C₁₀ alkyl, C₁-C₁₀ aminoalkyl, and C₆-C₂₀ aminoaryl, wherein R⁴ is selected from the group consisting of hydrogen, —(CH)COOH, —CHCH(CH)COOH, imidazoline, C₁-C₁₀ alcohol, C₁-C₁₀ ester, C₁-C₁₀ amide, and C₁-C₁₀ alkyl-C₆-C₂₀ aryl.

In some embodiments R¹, R², and R³ are each hydrogen, wherein n is 0, and wherein R⁴ is hydroxyethyl.

In certain aspects, the imidazoline is selected from the group consisting of tall oil hydroxyethyl imidazoline (CAS No. 61791-39-7) and oleic imidazoline (CAS No. 68052-47-1). In certain aspects, the imidazoline is tall oil hydroxyethyl imidazoline.

In some aspects the phthalate comprises a compound of formula (II);

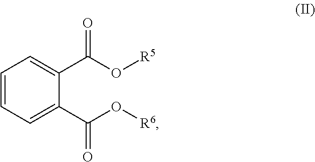

wherein R⁵ and R⁶ are each independently selected from the group consisting of C₁-C₁₀ alkyl, C₂-C₁₀ alkenyl, C₆-C₁₀ aryl, C₁-C₁₀ alkyl-C₆-C₁₀ aryl, C₆-C₁₀ aryl-C₁-C₁₀ alkyl, C₁-C₁₀ aminoalkyl, and C₆-C₁₀ aminoaryl.

In certain aspects, the phthalate is selected from the group consisting of dimethyl phthalate, diethyl phthalate, and diisobutyl phthalate. In certain aspects, the phthalate is dimethyl phthalate.

In some aspects, the acylamide comprises a compound of formula (III):

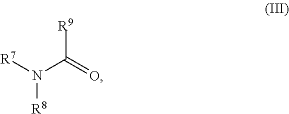

wherein R⁷, R³ and R⁹ are each independently selected from the group consisting of hydrogen, C₁-C₂₀ alkyl, C₂-C₂₀ alkenyl, and C₆-C₂₀ aryl.

In certain aspects, the acylamide is selected from the group consisting of dimethylacetamide and N,N-dimethylformamide. In certain aspects, the acylamide is dimethylacetamide.

In some embodiments, the compositions of the disclosure demonstrate synergy with respect to their ability to mitigate fouling. For example, in some embodiments, the compositions of the disclosure demonstrate greater fouling mitigation than the individual components present within the composition, controlling for the dosage of the active components.

In some embodiments, the compositions of the disclosure are active even under acidic conditions, unlike conventional polymerization inhibitors known in the art. Thus, in some embodiments, the compositions of the disclosure also include one or more acids.

Methods of mitigating fouling caused by polymerization of a monomer are also disclosed herein. The methods mitigating fouling caused by polymerization of a monomer include the step of adding a composition of the disclosure to the monomer. Addition of the compositions of the present disclosure to the process equipment can mitigate fouling.

The foregoing has outlined rather broadly the features and technical advantages of the present disclosure in order that the detailed description that follows may be better understood. Additional features and advantages of the disclosure will be described hereinafter that form the subject of the claims of this application. It should be appreciated by those skilled in the art that the conception and the specific embodiments disclosed may be readily utilized as a basis for modifying or designing other embodiments for carrying out the same purposes of the present disclosure. It should also be realized by those skilled in the art that such equivalent embodiments do not depart from the spirit and scope of the disclosure as set forth in the appended claims.

### DETAILED DESCRIPTION

Various embodiments of the present disclosure are described below. The relationship and functioning of the various elements of the embodiments may better be understood by reference to the following detailed description. However, embodiments are not limited to those explicitly described herein.

The present disclosure relates to compositions that include a blend of antifouling agents and methods of using the same to mitigate fouling caused by the polymerization of ethylenically unsaturated monomers. Antifouling compositions of the present disclosure include a dispersing agent comprising an imidazoline, a solvator comprising a phthalate and a promoter agent comprising an acylamide. The antifouling compositions can be blends of multiple components, including components in addition to the aforementioned compounds having a dispersing agent comprising an imidazoline, a solvator comprising a phthalate and a promoter agent comprising an acylamide.

### Definitions

The term “fouling” refers to the formation of polymers, prepolymers, oligomer and/or other materials, which would become insoluble in and/or precipitate from a stream and deposit on equipment under the conditions of operation of the equipment. In turn, the inhibitor, retarder, and amine stabilizer components and compositions of the disclosure can be referred to as “antifouling” as they inhibit or reduce such formation.

As used herein, “mitigating” or “mitigation” and the like mean that a composition does not inhibit polymer formation or fouling itself, but rather that it can transfer the formed foulant from the system to following units while as not blocking the pipeline.

### Compositions of the Disclosure

The present disclosure relates to compositions for mitigating fouling caused by monomer polymerization where the compositions include a dispersing agent comprising an imidazoline, a solvator comprising a phthalate and a promoter agent comprising an acylamide. In some embodiments, the compositions are for mitigating fouling caused by monomer polymerization, where the monomer is an ethylenically unsaturated monomer. For example, the compositions of the disclosure are useful for mitigating fouling caused by polymerization of ethylenically unsaturated monomers including, but not limited to, methacrylic acid, methyl methacrylate, acrylic acid, acrylic acid esters, methacrylamide sulfate, vinyl acetate, acrylonitrile, acrolein, acrylates, methacrylates, 1,3-butadiene, styrene, isoprene, and combinations thereof.

In some aspects, the dispersing agent comprising an imidazoline of formula (I):

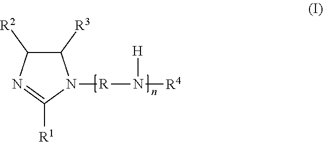

or a derivative thereof, wherein R is C₁-C₃₀ alkylene, wherein R¹, R² and R³ are each independently selected from the group consisting of H, C₁-C₃₀ alkyl, C₂-C₃₀ alkenyl, C₆-C₃₀ aryl, C₁-C₃₀ alkyl-C₆-C₃₀ aryl, C₆-C₃₀ aryl-C₁-C₃₀ alkyl, C₁-C₃₀ aminoalkyl, and C₆-C₃₀ aminoaryl, wherein R⁴ is selected from the group consisting of hydrogen, —CH₂COOH, —(CH₂)₂COOH, —CH₂CH(CH₃)COOH, imidazoline, C₁-C₃₀ alcohol, C₁-C₃₀ ester, C₁-C₃₀ amide, and C₁-C₃₀ alkylaryl, and wherein n is 0, 1, 2, 3, 4, 5, 6, 7, 8, or 9. In some embodiments, R is a C₁-C₁₀ alkylene.

In some embodiments, R is C₁-C₁₀ alkylene, wherein R¹, R² and R³ are each independently selected from the group consisting of H, C₁-C₁₀ alkyl, C₂-C₁₀ alkenyl, C₆-C₂₀ aryl, C₁-C₁₀ alkyl-C₆-C₂₀ aryl, C₆-C₂₀ aryl-C₁-C₁₀ alkyl, C₁-C₁₀ aminoalkyl, and C₆-C₂₀ aminoaryl, wherein R⁴ is selected from the group consisting of hydrogen, —(CH)COOH, —CHCH(CH)COOH, imidazoline, C₁-C₁₀ alcohol, C₁-C₁₀ ester, C₁-C₁₀ amide, and C₁-C₁₀ alkyl-C₆-C₂₀ aryl.

In some embodiments R¹, R², and R³ are each hydrogen, wherein n is 0, and wherein R⁴ is hydroxyethyl.

In certain aspects, the imidazoline is selected from the group consisting of tall oil hydroxyethyl imidazoline (CAS No. 61791-39-7) and oleic imidazoline (CAS No. 68052-47-1). In certain aspects, the imidazoline is tall oil hydroxyethyl imidazoline.

In some aspects the phthalate comprises a compound of formula (II);

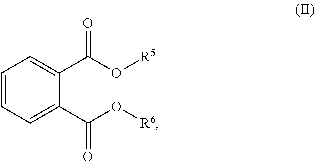

wherein R⁵ and R⁶ are each independently selected from the group consisting of C₁-C₁₀ alkyl, C₂-C₁₀ alkenyl, C₁-C₁₀ aryl, C₁-C₁₀ alkyl-C₆-C₁₀ aryl, C₆-C₁₀ aryl-C₁-C₁₀ alkyl, C₁-C₁₀ aminoalkyl, and C₆-C₁₀ aminoaryl.

In certain aspects, the phthalate is selected from the group consisting of dimethyl phthalate, diethyl phthalate, and diisobutyl phthalate. In certain aspects, the phthalate is dimethyl phthalate.

In some aspects the acylamide comprises a compound of formula (III):

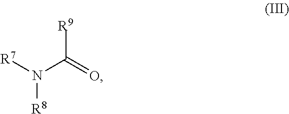

wherein R¹, R⁸ and R⁹ are each independently selected from the group consisting of hydrogen, C₁-C₂₀ alkyl, C₂-C₂₀ alkenyl, and C₁-C₂₀ aryl.

In certain aspects, the acylamide is selected from the group consisting of dimethylacetamide and N,N-dimethylformamide. In certain aspects, the acylamide is dimethylacetamide.

In some embodiments, the composition unexpectedly demonstrates synergy, where the combination of the a dispersing agent that is an imidazoline, a solvator that is a phthalate and a promoter agent that is an acylamide produces a greater degree of fouling mitigation than would be expected for the combination.

In some embodiments, the promoter agent is present in the composition at a concentration of about 0.1% to about 99.9% by weight. By way of example, but not limitation, the promoter agent can be present in the composition at a concentration about 0.1% to about 99.9%, about 0.1% to about 99%, about 0.1% to about 95%, about 0.1% to about 90%, about 0.1% to about 80%, about 0.1% to about 75%, about 0.1% to about 70%, about 0.1% to about 60%, about 0.1% to about 50%, about 0.1% to about 40%, about 0.1% to about 30%, about 0.1% to about 25%, about 0.1% to about 20%, about 0.1% to about 15%, about 0.1% to about 10% about 0.1% to about 5%, about 0.1% to about 1%, about 1% to about 99.9%, about 1% to about 99%, about 1% to about 95%, about 1% to about 90%, about 1% to about 80%, about 1% to about 75%, about 1% to about 70%, about 1% to about 60%, about 1% to about 50%, about 1% to about 40%, about 1% to about 30%, about 1% to about 25%, about 1% to about 20%, about 1% to about 15%, about 1% to about 10% about 1% to about 5%, about 5% to about 99%, about 5% to about 95%, about 5% to about 90%, about 5% to about 80%, about 5% to about 75%, about 5% to about 70%, about 5% to about 60%, about 5% to about 50%, about 5% to about 40%, about 5% to about 30%, about 5% to about 25%, about 5% to about 20%, about 5% to about 15%, about 5% to about 10%, about 10% to about 99.9%, about 10% to about 99%, about 10% to about 95%, about 10% to about 90%, about 10% to about 80%, about 10% to about 75%, about 10% to about 70%, about 10% to about 60%, about 10% to about 50%, about 10% to about 40%, about 10% to about 30%, about 10% to about 25%, about 10% to about 20%, about 10% to about 15%, about 15% to about 99.9%, about 15% to about 99%, about 15% to about 95%, about 15% to about 90%, about 15% to about 80%, about 15% to about 75%, about 15% to about 70%, about 15% to about 60%, about 15% to about 50%, about 15% to about 40%, about 15% to about 30%, about 15% to about 25%, about 15% to about 20%, about 20% to about 99.9%, about 20% to about 99%, about 20% to about 95%, about 20% to about 90%, about 20% to about 80%, about 20% to about 75%, about 20% to about 70%, about 20% to about 60%, about 20% to about 50%, about 20% to about 40%, about 20% to about 30%, about 20% to about 25%, about 25% to about 99.9%, about 25% to about 99%, about 25% to about 95%, about 25% to about 90%, about 25% to about 80%, about 25% to about 75%, about 25% to about 70%, about 25% to about 60%, about 25% to about 50%, about 25% to about 40%, about 25% to about 30%, about 30% to about 99.9%, about 30% to about 99%, about 30% to about 95%, about 30% to about 90%, about 30% to about 80%, about 30% to about 75%, about 30% to about 70%, about 30% to about 60%, about 30% to about 50%, about 30% to about 40%, about 40% to about 99.9%, about 40% to about 99%, about 40% to about 95%, about 40% to about 90%, about 40% to about 80%, about 40% to about 75%, about 40% to about 70%, about 40% to about 60%, about 40% to about 50%, about 50% to about 99.9%, about 50% to about 99%, about 50% to about 95%, about 50% to about 90%, about 50% to about 80%, about 50% to about 75%, about 50% to about 70%, about 50% to about 60%, about 60% to about 99.9%, about 60% to about 99%, about 60% to about 95%, about 60% to about 90%, about 60% to about 80%, about 60% to about 70%, about 70% to about 99.9%, about 70% to about 99%, about 70% to about 95%, about 70% to about 90%, about 70% to about 80%, about 70% to about 75%, about 75% to about 99.9%, about 75% to about 99%, about 75% to about 95%, about 75% to about 90%, about 75% to about 80%, about 80% to about 99.9%, about 80% to about 99%, about 80% to about 95%, about 80% to about 90%, about 90% to about 99.9%, about 90% to about 99%, about 90% to about 95%, about 95% to about 99.9%, about 95% to about 99%, about 99% to about 99.9%, about 0.1%, 1%, 5%, 10%, 15%, 20%, 25%, 30%, 40%, 50%, 60%, 70%, 75%, 80%, 90%, 95%, 99%, or 99.9% by weight. In some embodiments, the promoter agent is present in the composition a concentration of about 10% to about 60% by weight.

In some embodiments, the dispersing agent is present in the composition at a concentration of about 0.1% to about 99.9% by weight. By way of example, but not limitation, the dispersing agent can be present in the composition at a concentration about 0.1% to about 99.9%, about 0.1% to about 99%, about 0.1% to about 95%, about 0.1% to about 90%, about 0.1% to about 80%, about 0.1% to about 75%, about 0.1% to about 70%, about 0.1% to about 60%, about 0.1% to about 50%, about 0.1% to about 40%, about 0.1% to about 30%, about 0.1% to about 25%, about 0.1% to about 20%, about 0.1% to about 15%, about 0.1% to about 10% about 0.1% to about 5%, about 0.1% to about 1%, about 1% to about 99.9%, about 1% to about 99%, about 1% to about 95%, about 1% to about 90%, about 1% to about 80%, about 1% to about 75%, about 1% to about 70%, about 1% to about 60%, about 1% to about 50%, about 1% to about 40%, about 1% to about 30%, about 1% to about 25%, about 1% to about 20%, about 1% to about 15%, about 1% to about 10% about 1% to about 5%, about 5% to about 99%, about 5% to about 95%, about 5% to about 90%, about 5% to about 80%, about 5% to about 75%, about 5% to about 70%, about 5% to about 60%, about 5% to about 50%, about 5% to about 40%, about 5% to about 30%, about 5% to about 25%, about 5% to about 20%, about 5% to about 15%, about 5% to about 10%, about 10% to about 99.9%, about 10% to about 99%, about 10% to about 95%, about 10% to about 90%, about 10% to about 80%, about 10% to about 75%, about 10% to about 70%, about 10% to about 60%, about 10% to about 50%, about 10% to about 40%, about 10% to about 30%, about 10% to about 25%, about 10% to about 20%, about 10% to about 15%, about 15% to about 99.9%, about 15% to about 99%, about 15% to about 95%, about 15% to about 90%, about 15% to about 80%, about 15% to about 75%, about 15% to about 70%, about 15% to about 60%, about 15% to about 50%, about 15% to about 40%, about 15% to about 30%, about 15% to about 25%, about 15% to about 20%, about 20% to about 99.9%, about 20% to about 99%, about 20% to about 95%, about 20% to about 90%, about 20% to about 80%, about 20% to about 75%, about 20% to about 70%, about 20% to about 60%, about 20% to about 50%, about 20% to about 40%, about 20% to about 30%, about 20% to about 25%, about 25% to about 99.9%, about 25% to about 99%, about 25% to about 95%, about 25% to about 90%, about 25% to about 80%, about 25% to about 75%, about 25% to about 70%, about 25% to about 60%, about 25% to about 50%, about 25% to about 40%, about 25% to about 30%, about 30% to about 99.9%, about 30% to about 99%, about 30% to about 95%, about 30% to about 90%, about 30% to about 80%, about 30% to about 75%, about 30% to about 70%, about 30% to about 60%, about 30% to about 50%, about 30% to about 40%, about 40% to about 99.9%, about 40% to about 99%, about 40% to about 95%, about 40% to about 90%, about 40% to about 80%, about 40% to about 75%, about 40% to about 70%, about 40% to about 60%, about 40% to about 50%, about 50% to about 99.9%, about 50% to about 99%, about 50% to about 95%, about 50% to about 90%, about 50% to about 80%, about 50% to about 75%, about 50% to about 70%, about 50% to about 60%, about 60% to about 99.9%, about 60% to about 99%, about 60% to about 95%, about 60% to about 90%, about 60% to about 80%, about 60% to about 70%, about 70% to about 99.9%, about 70% to about 99%, about 70% to about 95%, about 70% to about 90%, about 70% to about 80%, about 70% to about 75%, about 75% to about 99.9%, about 75% to about 99%, about 75% to about 95%, about 75% to about 90%, about 75% to about 80%, about 80% to about 99.9%, about 80% to about 99%, about 80% to about 95%, about 80% to about 90%, about 90% to about 99.9%, about 90% to about 99%, about 90% to about 95%, about 95% to about 99.9%, about 95% to about 99%, about 99% to about 99.9%, about 0.1%, 1%, 5%, 10%, 15%, 20%, 25%, 30%, 40%, 50%, 60%, 70%, 75%, 80%, 90%, 95%, 99%, or 99.9% by weight. In some embodiments, the dispersing agent is present in the composition at a concentration of about 30% to about 80% by weight.

In some embodiments, the solvator agent is present in the composition at a concentration of about 0.1% to about 99.9% by weight. By way of example, but not limitation, the solvator agent can be present in the composition at a concentration about 0.1% to about 99.9%, about 0.1% to about 99%, about 0.1% to about 95%, about 0.1% to about 90%, about 0.1% to about 80%, about 0.1% to about 75%, about 0.1% to about 70%, about 0.1% to about 60%, about 0.1% to about 50%, about 0.1% to about 40%, about 0.1% to about 30%, about 0.1% to about 25%, about 0.1% to about 20%, about 0.1% to about 15%, about 0.1% to about 10% about 0.1% to about 5%, about 0.1% to about 1%, about 1% to about 99.9%, about 1% to about 99%, about 1% to about 95%, about 1% to about 90%, about 1% to about 80%, about 1% to about 75%, about 1% to about 70%, about 1% to about 60%, about 1% to about 50%, about 1% to about 40%, about 1% to about 30%, about 1% to about 25%, about 1% to about 20%, about 1% to about 15%, about 1% to about 10% about 1% to about 5%, about 5% to about 99%, about 5% to about 95%, about 5% to about 90%, about 5% to about 80%, about 5% to about 75%, about 5% to about 70%, about 5% to about 60%, about 5% to about 50%, about 5% to about 40%, about 5% to about 30%, about 5% to about 25%, about 5% to about 20%, about 5% to about 15%, about 5% to about 10%, about 10% to about 99.9%, about 10% to about 99%, about 10% to about 95%, about 10% to about 90%, about 10% to about 80%, about 10% to about 75%, about 10% to about 70%, about 10% to about 60%, about 10% to about 50%, about 10% to about 40%, about 10% to about 30%, about 10% to about 25%, about 10% to about 20%, about 10% to about 15%, about 15% to about 99.9%, about 15% to about 99%, about 15% to about 95%, about 15% to about 90%, about 15% to about 80%, about 15% to about 75%, about 15% to about 70%, about 15% to about 60%, about 15% to about 50%, about 15% to about 40%, about 15% to about 30%, about 15% to about 25%, about 15% to about 20%, about 20% to about 99.9%, about 20% to about 99%, about 20% to about 95%, about 20% to about 90%, about 20% to about 80%, about 20% to about 75%, about 20% to about 70%, about 20% to about 60%, about 20% to about 50%, about 20% to about 40%, about 20% to about 30%, about 20% to about 25%, about 25% to about 99.9%, about 25% to about 99%, about 25% to about 95%, about 25% to about 90%, about 25% to about 80%, about 25% to about 75%, about 25% to about 70%, about 25% to about 60%, about 25% to about 50%, about 25% to about 40%, about 25% to about 30%, about 30% to about 99.9%, about 30% to about 99%, about 30% to about 95%, about 30% to about 90%, about 30% to about 80%, about 30% to about 75%, about 30% to about 70%, about 30% to about 60%, about 30% to about 50%, about 30% to about 40%, about 40% to about 99.9%, about 40% to about 99%, about 40% to about 95%, about 40% to about 90%, about 40% to about 80%, about 40% to about 75%, about 40% to about 70%, about 40% to about 60%, about 40% to about 50%, about 50% to about 99.9%, about 50% to about 99%, about 50% to about 95%, about 50% to about 90%, about 50% to about 80%, about 50% to about 75%, about 50% to about 70%, about 50% to about 60%, about 60% to about 99.9%, about 60% to about 99%, about 60% to about 95%, about 60% to about 90%, about 60% to about 80%, about 60% to about 70%, about 70% to about 99.9%, about 70% to about 99%, about 70% to about 95%, about 70% to about 90%, about 70% to about 80%, about 70% to about 75%, about 75% to about 99.9%, about 75% to about 99%, about 75% to about 95%, about 75% to about 90%, about 75% to about 80%, about 80% to about 99.9%, about 80% to about 99%, about 80% to about 95%, about 80% to about 90%, about 90% to about 99.9%, about 90% to about 99%, about 90% to about 95%, about 95% to about 99.9%, about 95% to about 99%, about 99% to about 99.9%, about 0.1%, 1%, 5%, 10%, 15%, 20%, 25%, 30%, 40%, 50%, 60%, 70%, 75%, 80%, 90%, 95%, 99%, or 99.9% by weight. In some embodiments, the solvator agent is present in the composition at a concentration of about 30% to about 80% by weight.

In some embodiments, a weight ratio of the dispersing agent to the solvator agent can be from about 10:1 to about 1:10. By way of example, but not limitation, the weight ratio of the dispersing agent to the solvator agent can be from about 10:1 to about 1:10, 10:1 to about 1:9, 10:1 to about 1:8, 10:1 to about 1:7, about 10:1 to about 1:6, about 10:1 to about 1:5, about 10:1 to about 1:4, about 10:1 to about 1:3, about 10:1 to about 1:2, about 10:1 to about 1:1, about 9:1 to about 1:10, about 9:1 to about 1:9, about 9:1 to about 1:8, about 9:1 to about 1:7, about 9:1 to about 1:6, about 9:1 to about 1:5, about 9:1 to about 1:4, about 9:1 to about 1:3, about 9:1 to about 1:2, about 9:1 to about 1:1, about 8:1 to about 1:10, about 8:1 to about 1:9, about 8:1 to about 1:8, about 8:1 to about 1:7, about 8:1 to about 1:6, about 8:1 to about 1:5, about 8:1 to about 1:4, about 8:1 to about 1:3, about 8:1 to about 1:2, about 8:1 to about 1:1, about 7:1 to about 1:10, about 7:1 to about 1:9, about 7:1 to about 1:8, about 7:1 to about 1:7, about 7:1 to about 1:6, about 7:1 to about 1:5, about 7:1 to about 1:4, about 7:1 to about 1:3, about 7:1 to about 1:2, about 7:1 to about 1:1, about 6:1 to about 1:10, about 6:1 to about 1:9, about 6:1 to about 1:8, about 6:1 to about 1:7, about 6:1 to about 1:6, about 6:1 to about 1:5, about 6:1 to about 1:4, about 6:1 to about 1:3, about 6:1 to about 1:2, about 6:1 to about 1:1, about 5:1 to about 1:10, about 5:1 to about 1:9, about 5:1 to about 1:8, about 5:1 to about 1:7, about 5:1 to about 1:6, about 5:1 to about 1:5, about 5:1 to about 1:4, about 5:1 to about 1:3, about 5:1 to about 1:2, about 5:1 to about 1:1, about 4:1 to about 1:10, about 4:1 to about 1:9, about 4:1 to about 1:8, about 4:1 to about 1:7, about 4:1 to about 1:6, about 4:1 to about 1:5, about 4:1 to about 1:4, about 4:1 to about 1:3, about 4:1 to about 1:2, about 4:1 to about 1:1, about 3:1 to about 1:10, about 3:1 to about 1:9, about 3:1 to about 1:8, about 3:1 to about 1:7, about 3:1 to about 1:6, about 3:1 to about 1:5, about 3:1 to about 1:4, about 3:1 to about 1:3, about 3:1 to about 1:2, about 3:1 to about 1:1, about 2:1 to about 1:10, about 2:1 to about 1:9, about 2:1 to about 1:8, about 2:1 to about 1:7, about 2:1 to about 1:6, about 2:1 to about 1:5, about 2:1 to about 1:4, about 2:1 to about 1:3, about 2:1 to about 1:2, about 2:1 to about 1:1, about 1:1 to about 1:10, about 1:1 to about 1:9, about 1:1 to about 1:8, about 1:1 to about 1:7, about 1:1 to about 1:6, about 1:1 to about 1:5, about 1:1 to about 1:4, about 1:1 to about 1:3, about 1:1 to about 1:2, about 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, or 1:10.

The composition may optionally also include one or more additional solvents. In certain embodiments, the additional solvent can be selected from water, an ester, an aromatic, an aliphatic, an amide, an acylamide, an alcohol, a liquid polymer, and combinations thereof. Where an alcohol is used, the alcohol may be the same as is used in the manufacturing process such as, by way of example, but not limitation, in the (meth)acrylate manufacturing process.

In some embodiments, the composition also includes one or more ethylenically unsaturated monomers. One of ordinary skill in the art will appreciate that there are many ethylenically unsaturated monomers that are compatible with the compositions of the disclosure. For example, in some embodiments, the one or more ethylenically unsaturated monomers are selected from methyl methacrylate, acrylic acid, acrylic acid esters, methacrylamide sulfate, acrolein, acrylates, methacrylates, vinyl acetate, acrylonitrile, acrylates, methacrylates, 1,3-butadiene, styrene, isoprene, methacrylic acid, and combinations thereof. In certain embodiments, the composition also includes vinyl acetate. In certain embodiments, the composition also includes acrylonitrile. In certain embodiments, the composition also includes acrylates. In certain embodiments, the composition also includes methacrylates. In certain embodiments, the composition also includes 1,3-butadiene. In certain embodiments, the composition also includes styrene. In certain embodiments, the composition also includes isoprene. In certain embodiments, the composition also includes acrylic acid and/or methacrylic acid.

In some embodiments, the composition does not include (excludes) a phenol, a nitroxyl radical, a nitroso group compound, or hydroquinone.

The compositions of the disclosure are stable and remain useful fouling mitigators even under acidic conditions. Thus, the compositions of the disclosure are useful for mitigating polymer fouling during manufacturing process, particularly those that are performed under acidic conditions. For example, the compositions of the disclosure are useful for preventing polymerization of acrylates, which may include, but are not limited to, acrylonitrile, acrylic acid, methyl methacrylic acid and its esters, and vinyl acetate.

In certain embodiments, the compositions of the disclosure are generally stable under acidic conditions. Thus, in some embodiments, the composition also includes one or more acids. For example, in some embodiments, the composition also includes one or more acids selected from the group consisting of mineral acids and carboxylic acids. Mineral acids include, but are not limited to, hydrochloric acid, hydrofluoric acid, hydrobromic acid, hydroiodic acid, nitric acid, phosphoric acid, sulfuric acid, boric acid, perchloric acid, and the like. Carboxylic acids include, but are not limited to, formic acid, acetic acid, propionic acid, butyric acid, valeric acid, caproic acid, ethanic acid, caprylic acid, undecylic acid, lauric acid, oxalic acid, acrylic acid, substituted acrylic acid, malonic acid, succinic acid, glutaric acid, adipic acid, pimelic acid, suberic acid, and the like.

In some embodiments, the compositions of the disclosure also include hydrochloric acid. In some embodiments, the compositions of the disclosure also include nitric acid. In some embodiments, the compositions of the disclosure also include phosphoric acid. In some embodiments, the compositions of the disclosure also include sulfuric acid. In some embodiments, the compositions of the disclosure also include acetic acid. In some embodiments, the compositions of the disclosure also include propionic acid. In some embodiments, the compositions of the disclosure also include butyric acid. In some embodiments, the compositions of the disclosure also include valeric acid.

In some embodiments, the composition can be combined with or used with additional antifouling agents, such as hydroquinone, or inhibitors, such as HTEMPO and its derivatives, or phenothiazine, a passivator, a cleaning agent, a transition metal salt, an antioxidizer or a defoamer, which can be injected together or separately in methods of the disclosure.

### Methods of Using the Compositions of the Disclosure

The present disclosure also relates to methods of mitigating fouling caused by polymerization of monomers that include adding a composition of the disclosure to the monomer. In some aspects, an effective amount of the composition of the disclosure is added to the monomer, where an effective amount is any amount sufficient to mitigate fouling caused by polymerization of the monomer.

In some aspects, the monomer is an ethylenically unsaturated monomer. In some aspects, the monomer is an ethylenically unsaturated monomer selected from vinyl acetate, acrylonitrile, acrylates, methacrylates, 1,3-butadiene, styrene, isoprene, (meth)acrylic acid, and combinations thereof are disclosed. In some aspects, the methods disclosed herein are useful in inhibiting the polymerization of vinyl acetate. In some aspects, the methods disclosed herein are useful in inhibiting the polymerization of acrylonitrile. In some aspects, the methods disclosed herein are useful in inhibiting the polymerization of acrylates. In some aspects, the methods disclosed herein are useful in inhibiting the polymerization of methacrylates. In some aspects, the methods disclosed herein are useful in inhibiting the polymerization of 1,3-butadiene. In some aspects, the methods disclosed herein are useful in inhibiting the polymerization of styrene. In some aspects, the methods disclosed herein are useful in inhibiting the polymerization of isoprene. In some aspects, the methods disclosed herein are useful in inhibiting the polymerization of methacrylic acid and/or acrylic acid.

The composition of the disclosure can be added manually or automatically to the fluid. The composition can also be added continuously and/or intermittently. Automatic addition may be accomplished through the use of chemical injection pumps. The chemical injection pumps may be programmed to add particular amounts of the polymerization inhibitor composition, or any components thereof, at certain time intervals to the fluid. In alternate aspects, the chemical injection pumps can be manually controlled to add particular amounts of the polymerization inhibitor composition, or any components thereof, to the fluid. Addition of the presently disclosed polymerization inhibitor compositions to the monomer will thereby inhibit polymerization of the monomer.

In some aspects, the monomer is provided as a neat liquid. In other aspects, the monomer is provided within a solution, hereafter referred to as “the monomer solution”.

In some aspects, the monomer solution also includes one or more additional components selected from an acid, an organic solvent, water, and combinations thereof. For example, in some aspects, the monomer solution includes one or more organic solvents selected from vinyl acetate, dimethyl phthalate, dimethylformamide, toluene, xylene, highly aromatic naphtha, acetonitrile, ethyl acetate, acetone, dichloromethane, tetrahydrofuran, hexanes, dimethyl sulfoxide, N-methyl-2-pyrrolidone, and combinations thereof. In some aspects, the monomer solution includes one or more acids selected from hydrochloric acid, hydrofluoric acid, hydrobromic acid, hydroiodic acid, nitric acid, phosphoric acid, sulfuric acid, boric acid, perchloric acid, formic acid, acetic acid, propionic acid, butyric acid, valeric acid, caproic acid, ethanic acid, caprylic acid, undecylic acid, lauric acid, oxalic acid, malonic acid, succinic acid, glutaric acid, adipic acid, pimelic acid, and suberic acid. In some aspects, the monomer solution includes water.

In some aspects, the monomer solution has a pH value of about 1 to about 7. In some aspects, the monomer solution has a pH value of about 1 to about 6. In some aspects, the monomer solution has a pH value of about 2 to about 6. In some aspects, the monomer solution has a pH value of about 3 to about 6. In some aspects, the monomer solution has a pH value of about 4 to about 6. In some aspects, the monomer solution has a pH value of about 5 to about 6.

In some aspects, the composition is added to the monomer such that the concentration of the composition as whole (dispersing agent, solvator agent and promoter agent) is between about 0.01 ppm and 10,000 ppm. By way of example, but not limitation, the concentration of the composition as a whole can be between about 0.01 ppm to about 10,000 ppm, about 0.01 ppm to about 5,000 ppm, about 0.01 ppm to about 4,000 ppm, about 0.01 ppm to about 3,000 ppm, about 0.01 ppm to about 2,000 ppm, about 0.01 ppm to about 1,000 ppm, about 0.01 ppm to about 500 ppm, about 0.01 ppm to about 100 ppm, about 0.01 ppm to about 50 ppm, about 0.01 ppm to about 40 ppm, about 0.01 ppm to about 30 ppm, about 0.01 ppm to about 20 ppm, about 0.01 ppm to about 10 ppm, about 0.01 ppm to about 5 ppm, about 0.01 ppm to about 1 ppm, about 0.1 ppm to about 10,000 ppm, about 0.1 ppm to about 5,000 ppm, about 0.1 ppm to about 4,000 ppm, about 0.1 ppm to about 3,000 ppm, about 0.1 ppm to about 2,000 ppm, about 0.1 ppm to about 1,000 ppm, about 0.1 ppm to about 500 ppm, about 0.1 ppm to about 100 ppm, about 0.1 ppm to about 50 ppm, about 0.1 ppm to about 40 ppm, about 0.1 ppm to about 30 ppm, about 0.1 ppm to about 20 ppm, about 0.1 ppm to about 10 ppm, about 0.1 ppm to about 5 ppm, about 0.1 ppm to about 1 ppm, about 1 ppm to about 10,000 ppm, about 1 ppm to about 5,000 ppm, about 1 ppm to about 4,000 ppm, about 1 ppm to about 3,000 ppm, about 1 ppm to about 2,000 ppm, about 1 ppm to about 1,000 ppm, about 1 ppm to about 500 ppm, about 1 ppm to about 100 ppm, about 1 ppm to about 50 ppm, about 1 ppm to about 40 ppm, about 1 ppm to about 30 ppm, about 1 ppm to about 20 ppm, about 1 ppm to about 10 ppm, about 1 ppm to about 5 ppm, about 5 ppm to about 10,000 ppm, about 5 ppm to about 5,000 ppm, about 5 ppm to about 4,000 ppm, about 5 ppm to about 3,000 ppm, about 5 ppm to about 2,000 ppm, about 5 ppm to about 1,000 ppm, about 5 ppm to about 500 ppm, about 5 ppm to about 100 ppm, about 5 ppm to about 50 ppm, about 5 ppm to about 40 ppm, about 5 ppm to about 30 ppm, about 5 ppm to about 20 ppm, about 5 ppm to about 10 ppm, about 10 ppm to about 10,000 ppm, about 10 ppm to about 5,000 ppm, about 10 ppm to about 4,000 ppm, about 10 ppm to about 3,000 ppm, about 10 ppm to about 2,000 ppm, about 10 ppm to about 1,000 ppm, about 10 ppm to about 500 ppm, about 10 ppm to about 100 ppm, about 10 ppm to about 50 ppm, about 10 ppm to about 40 ppm, about 10 ppm to about 30 ppm, about 10 ppm to about 20 ppm, about 20 ppm to about 10,000 ppm, about 20 ppm to about 5,000 ppm, about 20 ppm to about 4,000 ppm, about 20 ppm to about 3,000 ppm, about 20 ppm to about 2,000 ppm, about 20 ppm to about 1,000 ppm, about 20 ppm to about 500 ppm, about 20 ppm to about 100 ppm, about 20 ppm to about 50 ppm, about 20 ppm to about 40 ppm, about 20 ppm to about 30 ppm, about 30 ppm to about 10,000 ppm, about 30 ppm to about 5,000 ppm, about 30 ppm to about 4,000 ppm, about 30 ppm to about 3,000 ppm, about 30 ppm to about 2,000 ppm, about 30 ppm to about 1,000 ppm, about 30 ppm to about 500 ppm, about 30 ppm to about 100 ppm, about 30 ppm to about 50 ppm, about 30 ppm to about 40 ppm, about 40 ppm to about 10,000 ppm, about 40 ppm to about 5,000 ppm, about 40 ppm to about 4,000 ppm, about 40 ppm to about 3,000 ppm, about 40 ppm to about 2,000 ppm, about 40 ppm to about 1,000 ppm, about 40 ppm to about 500 ppm, about 40 ppm to about 100 ppm, about 40 ppm to about 50 ppm, about 50 ppm to about 10,000 ppm, about 50 ppm to about 5,000 ppm, about 50 ppm to about 4,000 ppm, about 50 ppm to about 3,000 ppm, about 50 ppm to about 2,000 ppm, about 50 ppm to about 1,000 ppm, about 50 ppm to about 500 ppm, about 50 ppm to about 100 ppm, about 100 ppm to about 10,000 ppm, about 100 ppm to about 5,000 ppm, about 100 ppm to about 4,000 ppm, about 100 ppm to about 3,000 ppm, about 100 ppm to about 2,000 ppm, about 100 ppm to about 1,000 ppm, about 100 ppm to about 500 ppm, about 500 ppm to about 10,000 ppm, about 500 ppm to about 5,000 ppm, about 500 ppm to about 4,000 ppm, about 500 ppm to about 3,000 ppm, about 500 ppm to about 2,000 ppm, about 500 ppm to about 1,000 ppm, about 1,000 ppm to about 10,000 ppm, about 1,000 ppm to about 5,000 ppm, about 1,000 ppm to about 4,000 ppm, about 1,000 ppm to about 3,000 ppm, about 1,000 ppm to about 2,000 ppm, about 2,000 to about 10,000 ppm, about 2,000 ppm to about 5,000 ppm, about 2,000 ppm to about 4,000 ppm, about 2,000 ppm to about 3,000 ppm, about 3,000 ppm to about 10,000 ppm, about 3,000 ppm to about 5,000 ppm, about 3,000 ppm to about 4,000 ppm, about 4,000 ppm to about 10,000 ppm, about 4,000 ppm to about 5,000 ppm, about 5,000 ppm to about 10,000 ppm, about 0.01, 0.1, 1, 5, 10, 20, 30, 40, 50, 100, 500, 1,000, 2,000, 3,000, 4,000, or 5,000 ppm.

The methods of the disclosure are useful for mitigating fouling caused by premature polymerization of monomers during manufacturing process, particularly those that are performed under acidic conditions. For example, the methods of the disclosure are useful for mitigating fouling caused by polymerization of acrylates, which may include, but are not limited to, acrylonitrile, acrylic acid, methyl methacrylic acid and its esters, and vinyl acetate.

### Processes for Preparing Compositions of the Disclosure

Compositions of the disclosure can be prepared by blending. Alternatively, the compositions can be prepared in situ by adding the each component to the process equipment. Relevant manufacturing process can include, by way of example but not limitation, methods to produce methyl methacrylate, such as the ACH process of the oxidation process which can divided into 4 units—the methyl methacrylate production unit, esterification unit, purification unit, and spent acid unit. The compositions of the disclosure or components thereof can be injected separately or together.

In the ACH manufacturing process, there are 3 reactive monomers—methacrylic acid, methacrylamide sulfate, and methyl methacylate. All of these monomers are easily polymerized to generate fouling issues and usually the polymerization occurs at the same location, resulting in a complex and variable fouling issue. The fouling issue often occurs in the esterification unit, purification unit, and spent acid unit.

In some embodiments, additional antifouling chemicals can be added to the reactor including antioxidants, hydroquinone, phenothiazine, tetramethylpiperidinooxy, inorganic salts, phenylenediamine and the like.

### EXAMPLES

### Example 1—Miscibility Testing

Dimethyl phthalate (DMP) was mixed with tall oil hydroxyethyl imidazoline in a 50/50 mixture and shaken to mix the two components. The tube was allowed to sit for 2 days, after which the two components had separated into separate layers.

45% DMP, 45% tall oil hydroxyethyl imidazoline and 10% dimethacetamide were mixed the same way and allowed to sit and no separation was observed.

### Example 2—Foulant Dissolution Test

A particle (0.3 g) of foulant was added to 15 mL of 30% dimethacetamide/70% DMP, DMP alone, or 30% dimethacetamide/70% imidazoline in test tubes. After 3 days standing at room temperature, the foulant had partially dissolved in the samples containing dimethacetamide but not in the DMP only sample, indicating that the dimethacetamide improves the efficiency of the DMP and imidazoline for dissolving foulant.

A particle (0.5 g) of asphaltene foulant was added to 15 mL of 30% dimethacetamide/70% DMP, 50% dimethacetamide/50% DMP, DMP alone, or 30% dimethacetamide/70% imidazoline in test tubes. After 3 days standing at room temperature, the foulant was not significantly dissolved in DMP, but was partially dissolved in the other samples, indicating that the dimethacetamide improves the efficiency of the DMP and imidazoline for dissolving foulant.

### Example 3—Dispersion Test

0.5 mL of foulant liquid was added into 10 mL methyl methacrylate to simulate a process stream. 50 μL of 20% dimethacetamide/80% DMP, 20% dimethacetamide/80% imidazoline, or 20% dimethacetamide/40% DMP/40% imidazoline was added to separate tubes containing the foulant liquid and methyl methacrylate. The mixtures were then mixed and allowed to stand for 10 minutes. Dimethacetamide/DMP was limited in its dispersion performance with most of the foulant deposit at the bottom while the foulant was suspended more in the liquid for the other two samples. The foregoing results demonstrate that, while DMP is a solvent for foulant and imidazoline is a dispersant, the two do not mix well without dimethacetamide, which acts as a solvent booster for the foulant, enabling quick dissolution and suspension of the foulant in the process stream.

All of the compositions and methods disclosed and claimed herein can be made and executed without undue experimentation in light of the present disclosure. While this invention may be embodied in many different forms, there are described in detail herein specific preferred embodiments of the invention. The present disclosure is an exemplification of the principles of the invention and is not intended to limit the invention to the particular embodiments illustrated. In addition, unless expressly stated to the contrary, use of the term “a” is intended to include “at least one” or “one or more.” For example, “a compound” is intended to include “at least one compound” or “one or more compounds.”

Any ranges given either in absolute terms or in approximate terms are intended to encompass both, and any definitions used herein are intended to be clarifying and not limiting. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges (including all fractional and whole values) subsumed therein.

Any composition disclosed herein may comprise, consist of, or consist essentially of any element, component and/or ingredient disclosed herein or any combination of two or more of the elements, components or ingredients disclosed herein.

Any method disclosed herein may comprise, consist of, or consist essentially of any method step disclosed herein or any combination of two or more of the method steps disclosed herein.

The transitional phrase “comprising,” which is synonymous with “including,” “containing,” or “characterized by,” is inclusive or open-ended and does not exclude additional, un-recited elements, components, ingredients and/or method steps.

The transitional phrase “consisting of” excludes any element, component, ingredient, and/or method step not specified in the claim.

The transitional phrase “consisting essentially of” limits the scope of a claim to the specified elements, components, ingredients and/or steps, as well as those that do not materially affect the basic and novel characteristic(s) of the claimed invention.

Unless specified otherwise, all molecular weights referred to herein are weight average molecular weights and all viscosities were measured at 25° C. with neat (not diluted) polymers.

As used herein, the term “about” refers to the cited value being within the errors arising from the standard deviation found in their respective testing measurements, and if those errors cannot be determined, then “about” may refer to, for example, within 5%, 4%, 3%, 2%, or 1% of the cited value.

Furthermore, the invention encompasses any and all possible combinations of some or all of the various embodiments described herein. It should also be understood that various changes and modifications to the presently preferred embodiments described herein will be apparent to those skilled in the art. Such changes and modifications can be made without departing from the spirit and scope of the invention and without diminishing its intended advantages. It is therefore intended that such changes and modifications be covered by the appended claims.

What is claimed is:

1. A composition for mitigating monomer polymerization, comprising:
a dispersing agent comprising an imidazoline or a derivative thereof;
a solvator agent comprising a phthalate or a derivative thereof; and
a promoter agent comprising a acylamide or a derivative thereof.

2. The composition of claim 1, wherein the imidazoline comprises a compound of formula (I): 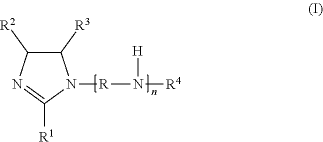
wherein
R is C₁-C₃₀ alkylene;
R¹, R² and R³ are each independently selected from the group consisting of H, C₁-C₃₀ alkyl, C₂-C₃₀ alkenyl, C₆-C₃₀ aryl, C₁-C₃₀ alkyl-C₆-C₃₀ aryl, C₆-C₃₀ aryl-C₁-C₃₀ alkyl, C₁-C₃₀ aminoalkyl, and C₆-C₃₀ aminoaryl;
R⁴ is selected from the group consisting of hydrogen, —CH₂COOH, —(CH₂)₂COOH, —CH₂CH(CH₃)COOH, imidazoline, alcohol, C₁-C₃₀ ester, C₁-C₃₀ amide, and C₁-C₃₀ alkyl-C₆-C₃₀ aryl; and
n is 1, 2, 3, 4, 5, 6, 7, 8, or 9.

3. The composition of claim 2, wherein R¹, R² and R³ are each hydrogen, wherein n is 0, and wherein R⁴ is hydroxyethyl.

4. The composition of claim 2, wherein R is C₁-C₁₀ alkylene.

5. The composition of claim 1, wherein the imidazoline is selected from the group consisting of tall oil hydroxyethyl imidazoline and oleic imidazoline.

6. The composition of claim 1, wherein the phthalate comprises a compound of formula (II): 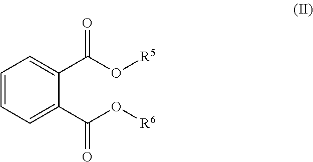
wherein R⁵ and R⁶ are each independently selected from the group consisting of C₁-C₁₀ alkyl, C₂-C₁₀ alkenyl, C₆-C₁₀ aryl, C₁-C₁₀ alkyl-C₆-C₁₀ aryl, C₆-C₁₀ aryl-C₁-C₁₀ alkyl, C₁-C₁₀ aminoalkyl, and C₆-C₁₀ aminoaryl.

7. The composition of claim 1, wherein the phthalate is selected from the group consisting of dimethyl phthalate, diethyl phthalate, and diisobutyl phthalate.

8. The composition of claim 1, wherein the acylamide comprises a compound of formula (III): 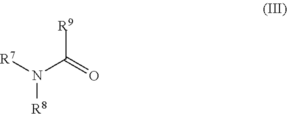
wherein R⁷, R³ and R⁹ are each independently selected from the group consisting of hydrogen, C₁-C₂₀ alkyl, C₂-C₂₀ alkenyl, and C₆-C₂₀ aryl.

9. The composition of claim 1, wherein the acylamide is selected from the group consisting of dimethylacetamide and N,N-dimethylformamide.

10. The composition of claim 1, wherein the promoter agent is present in the composition at a concentration of about 0.1% by weight to about 99.9% by weight.

11. The composition of claim 1, wherein the dispersing agent and the solvator agent are present at a concentration of about 0.1% by weight to about 99.9% by weight.

12. The composition of claim 1, wherein a weight ratio of the dispersing agent to the solvator agent can be from about 1:10 to about 10:1.

13. The composition of claim 1, further comprising a monomer.

14. The composition of claim 13, wherein the monomer is an ethylenic unsaturated monomer.

15. The composition of claim 13, wherein the monomer is methacrylic acid, methyl methacrylate, methacrylamide sulfate, acrylic acid, an acrylic acid ester, vinyl acetate, or any combination thereof.

16. The composition of claim 1, further comprising a polymerization inhibitor or an antioxidant.

17. A method of inhibiting polymerization of a monomer, the method comprising:
adding the composition of claim 1 to the monomer.

18. The method of claim 17, wherein the monomer is provided within a solution, wherein the solution further comprises an additional component selected from an acid, an organic solvent, water, and any combination thereof.

19. The method of claim 17, wherein the monomer is an ethylenic unsaturated monomer.

20. The method of claim 17, wherein the composition is added to the monomer such that a concentration of the composition is from about 0.01 ppm to about 10,000 ppm.
